(19) United States
(12) Patent Application Publication
(10) Pub. No.: US 2011/0071380 A1
(43) Pub. Date: Mar. 24, 2011

(54) Manual Instrumented Medical Tool System

(76) Inventors: Andrew A. GOLDENBERG, TORONTO (CA); John TRACHTENBERG, TORONTO (CA); Yi YANG, TORONTO (CA); Liang MA, TORONTO (CA)

(21) Appl. No.: 12/878,840

(22) Filed: Sep. 9, 2010

Related U.S. Application Data
(60) Provisional application No. 61/272,296, filed on Sep. 9, 2009.

(51) Int. Cl. A61B 5/055; A61B 6/00

(52) U.S. Cl. 600/411; 600/424

(57) ABSTRACT

A medical device is for use in association with a medical image of the gland or organ having a known reference point. The medical device comprises a structural frame, a horizontal joint, a vertical joint, a pan join, a tilt joint a medical instrument assembly and a control system. The medical device is positioned at a predetermined location relative to the medical image reference point. Each of the horizontal joint, the vertical joint, the pan joint and the tilt joint have a position sensor and are operably connected to the frame. The medical instrument assembly is operably connected to a sensor and to the horizontal joint, the vertical joint, the pan joint and the tilt joint. The control system is operably connected to the other elements whereby the control system determines the position of a predetermined location on the medical instrument assembly relative to the structural frame.

### CROSS REFERENCE TO RELATED PATENT APPLICATION

This patent application relates to U.S. Provisional Patent Application Ser. No. 61/272,296 filed on Sep. 9, 2009 entitled MANUAL INSTRUMENTED MEDICAL TOOL SYSTEM which is incorporated herein by reference in its entirety.

### FIELD OF THE INVENTION

This invention relates to tools for use in surgery and in particular manual tools that may be used for Minimally Invasive Surgery (MIS) such as prostate-related interventions: focal ablation, brachytherapy, and biopsy

### BACKGROUND OF THE INVENTION

The localized treatment of tumors and other medical conditions can be performed by: (i) focal ablation, coagulation of diseased tissue; and (ii) brachytherapy, the implantation of radioactive materials. Focal ablation is used to heat the tissue locally until it coagulates thus destroying the tumor cells. Implantation of radioactive implants directly into tumors results also in the destruction of the tumor cells. These types of surgeries are used for prostate therapy. An additional intervention is biopsy, a method of diagnosis of cancer.

One particular challenge with these types of surgeries is for the surgeon, during surgery, to know the location of the end of the interventional (surgical) needle with respect to the tumor, that is, the location of the element that comes into contact with the tumor, and provides the anatomical changes thereof.

Accordingly it would be advantageous to provide a method of locating the tip of the surgical instrument (needle) in real time and displaying that location on images of the organ or gland being surgically affected. Such medical images are obtained by ultrasound, or other type of imaging process such as MR (magnetic resonance).

### SUMMARY OF THE INVENTION

The present invention relates to a medical device for use in association with a medical image of the gland or organ having a known reference point, the medical device comprising: a structural frame being positioned at a predetermined (and measurable) location relative to the medical image reference point; a horizontal joint operably connected to a horizontal position sensor and operably connected to the frame; a vertical joint operably connected to a vertical position sensor and operably connected to the frame; a pan joint operably connected to a pan position sensor and operably connected to the frame; a tilt joint operably connected a tilt position sensor and operably connected to the frame; a medical instrument assembly operably connected to a medical instrument position sensor and operably connected to the horizontal joint, the vertical joint, the pan joint and the tilt joint; a control system operably connected to the horizontal position sensor, the vertical position sensor, the pan position sensor, the tilt position sensor, the tilt position sensor and the medical instrument position sensor whereby the control system determines the position of a predetermined location on the medical instrument assembly relative to the structural frame.

The medical device may further include a mover being positioned at a predetermined location relative to the medical image reference point, wherein the frame is movably attached to the mover and may further include a means for determining the position of the frame relative to the mover such that the position of the frame is positioned at a predetermined location relative to the medical image reference point.

The horizontal joint and horizontal position sensor of the medical device may include a multi-turn potentiometer operably connected to an anti-backless spur gear and a rack, a linear guide unit operably connected to the rack, a locking mechanism operably connected to the rack and a means for moving the rack operably connected to the rack.

The vertical joint and horizontal position sensor of the medical device may include a multi-turn potentiometer operably connected to an anti-backless spur gear and a rack, a locking mechanism operably connected to the rack and a means for moving the rack operably connected to the rack.

The medical device as claimed in any one of claims 1 to 4 wherein the pan joint and pan position sensor includes a rotary potentiometer a pan joint support operably connected to the potentiometer and a locking mechanism operably connected to the potentiometer.

The tilt joint and tilt position sensor may include a rotary potentiometer a shaft operably connected to the potentiometer, a tilt joint support operably connected to the potentiometer and a locking mechanism.

The medical instrument assembly may be a needle assembly.

The needle assembly and medical instrument assembly position sensor may include a linear potentiometer, a needle tool operably connected to the linear potentiometer, a guiding shaft for receiving the needle tool, a lock operably connected to the guiding shaft, a slide block operably connected to the guiding shaft and a connector.

The medical image may be an ultrasound image or an MR image and it may be obtained in real time. Alternatively the medical image may be a blended real time ultrasound image and a pre-operative MR image.

In another aspect of the invention there is provided a method of positioning a medical instrument assembly comprising the steps of:

obtaining a magnetic resonance image of the organ or gland;

obtaining an ultrasound image of the organ or gland;

merging the magnetic resonance image with the ultrasound image to obtain a merged image;

determining a position of a predetermined point on the medical instrument assembly connected to a manual medical tool system; and

locating the position of the predetermined point on the merged image.

The position of the predetermined point of the medical instrument may be determined continuously in real time and a location of the point may move on the merged image as the medical instrument assembly moves.

The ultrasound image may be obtained continuously in real time.

The method may further include the step of determining a best path to reach a predetermined target in order to move the medical instrument and show the best path on the merged image.

In a further aspect of the invention a method of positioning a medical instrument assembly including a medical instrument comprises the steps of:

obtaining a magnetic resonance image;

determining a position of a predetermined point on the medical instrument assembly connected to a manual medical tool system; and

locating the position of the predetermined point on the magnetic resonance image.

The position of the predetermined point of the medical instrument may be determined continuously in real time and a location of the point may move on the magnetic resonance image as the medical instrument assembly moves.

The magnetic resonance image may be updated as the medical instrument is being moved.

The method may further include the step of determining a best path based to move the medical instrument and showing the best path on the magnetic resonance image.

The method may be used in association with minimally invasive surgery and the minimally invasive surgery may be chosen from the group consisting of focal ablation, brachytherapy and biopsy.

Further features of the invention will be described or will become apparent in the course of the following detailed description.

### BRIEF DESCRIPTION OF THE DRAWINGS

The invention will now be described by way of example only, with reference to the accompanying drawings, in which:

FIG. 1 is a schematic representation of the manual instrumented focal ablation tool (MIFAT) system architecture of the present invention;

FIG. 2 is a side view of the manual instrumented focal ablation tool mounted on a stepper with a probe attached thereto;

FIG. 3 is a side view similar to that shown in FIG. 2 but also showing the patient and the needle assembly;

FIG. 4 is a perspective view of the manual instrumented focal ablation tool constructed in accordance with the present invention;

FIG. 5 is a perspective view of the horizontal and vertical movements portion of the manual instrumented focal ablation tool shown in FIG. 4;

FIG. 6 is a perspective view of the pan and tilt joints of the manual instrumented focal ablation tool shown in FIG. 4;

FIG. 7 is a perspective view of the needle assembly of the manual instrumented focal ablation tool shown in FIG. 4;

FIG. 8 is a perspective view of the stepper linear position sensing portion of the manual instrumented focal ablation tool shown in FIG. 4;

FIG. 9 is a diagram showing the electrical circuit for determining the measurement of needle position;

FIG. 10 is a view of a portion of the video screen which includes the video control area;

FIG. 11 is a view of a portion of the video screen which includes the sensor area;

FIG. 12 is a view of a portion of the video screen which includes the contour overlay area;

FIG. 13 is a view of a portion of the video screen which includes the best path area;

FIG. 14 is a perspective view of a prostate phantom;

FIG. 15 is a trans-rectal ultrasound image showing a transverse view with contouring of the prostate and the lesion;

FIG. 16 is a trans-rectal ultrasound image showing a screenshot of fused mri/trus guidance needle intervention;

FIG. 17 is a perspective view of an alternate embodiment of the manual instrumented focal ablation tool constructed in accordance with the present invention; and

FIG. 18 is a perspective view of the horizontal and vertical movement units of the alternate manual instrumented focal ablation tool shown in FIG. 17.

### DETAILED DESCRIPTION OF THE INVENTION

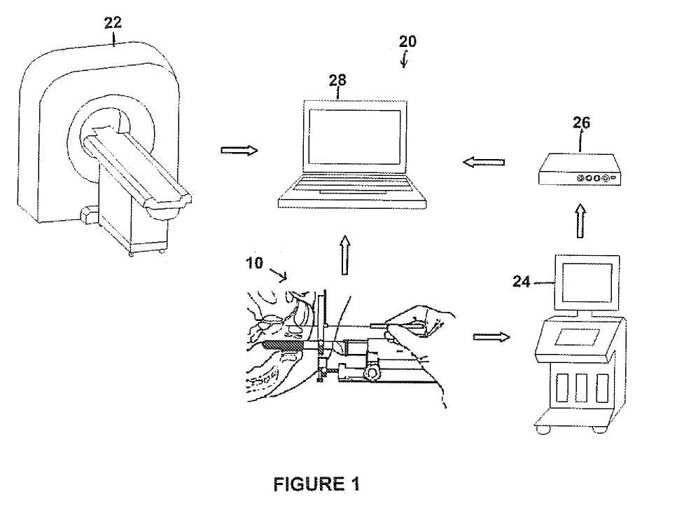

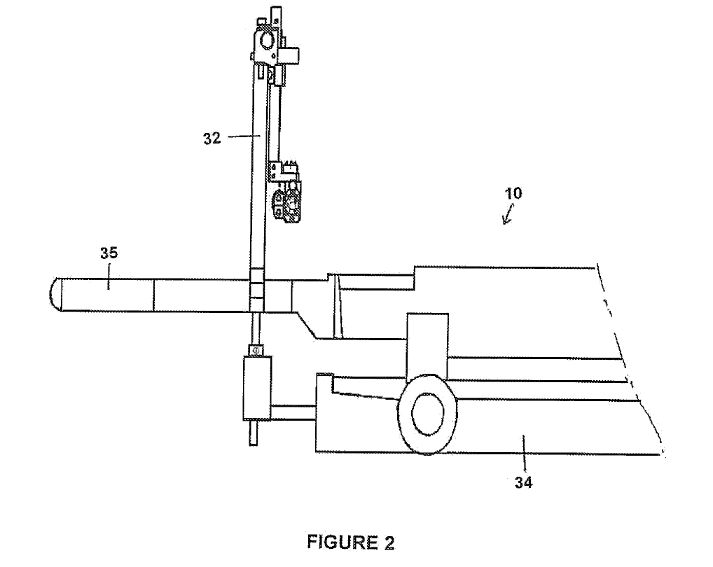

Referring to FIGS. 1 and 2, the manual instrumented focal ablation tool (MIFAT) of the present invention is adapted to be used in association with a TRUS (trans-rectal ultrasound) device including a probe positioning stepper with the combined MIFAT system shown generally at 10. The MIFAT system is adapted to be used in association with treatment planning and monitoring software system.

The MIFAT system architecture is shown in FIG. 1 at 20. The MIFAT system architecture includes the combined MIFAT and stepper with TRUS probe shown at 10, a pre-treatment magnetic resonance imaging 22, real-time ultrasound image 24, video capturer 26 and a computer with a graphical user interface 28.

The treatment planning and monitoring software system is comprised of the a plurality of modules namely: 1) MRI and ultrasound image-fusion; 2) real-time ultrasound image capture and the contour overlay display; 3) a treatment planning (the best path optimization for the needle insertion); 4) an image-registered intervention; 5) desired needle insertion overlay on real time ultrasonic image; 6) user graphical interface (GUI).

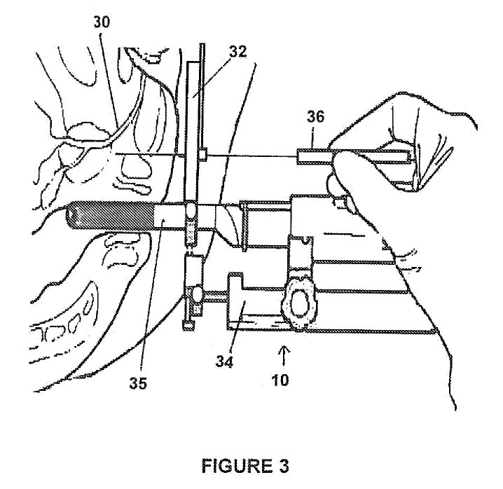

For intervention, the patient is placed on the standard Operation Room (OR) table. The combined MIFAT device and TRUS probe are secured to a mover of precision stepper that is attached on a precision stabilizer mounted on the operating room table. The precision stepper and precision stabilizer may be obtained from Radiation Therapy Products (RTP). FIG. 3 shows the position of a patient prostate 30, the MIFAT device 32, and the stepper 34 with TRUS probe 35 and a medical instrument assembly shown herein as assembly 36. The Instrumented Focal Ablation Tool (MIFAT) 32 is used to navigate the manual medical tool (needle) by manually controlling needle placements under trans-rectal ultrasound guidance overlaid on the pre-operational MR image.

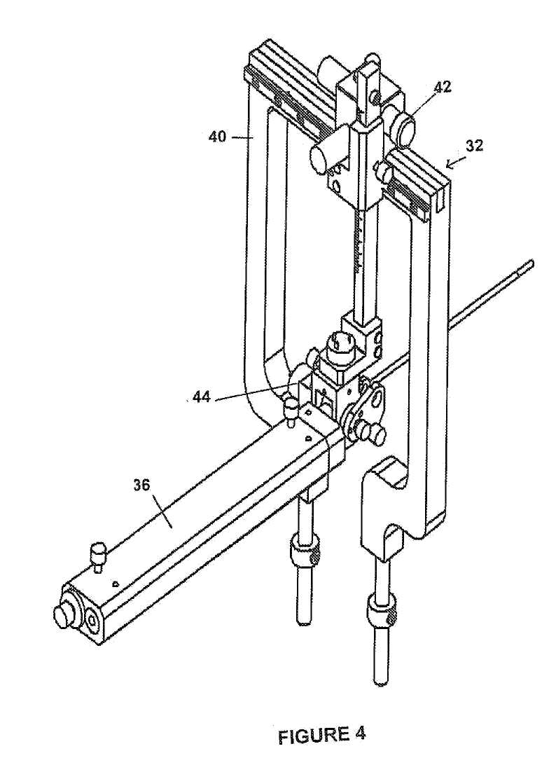

Referring to FIG. 4, the MIFAT device 32 consists of a frame 40, two linear motion joints 42 (horizontal and vertical), two rotational joints 44 (Pan and Tilt) and a medical instrument assembly 36. Each joint is electronically encoded (the displacement measurement is implemented by a potentiometer and fed back to computer through an analog-to-digital converter), so the position of each joint is always known by the computer. FIG. 4 provides a schematic overview of the tool device.

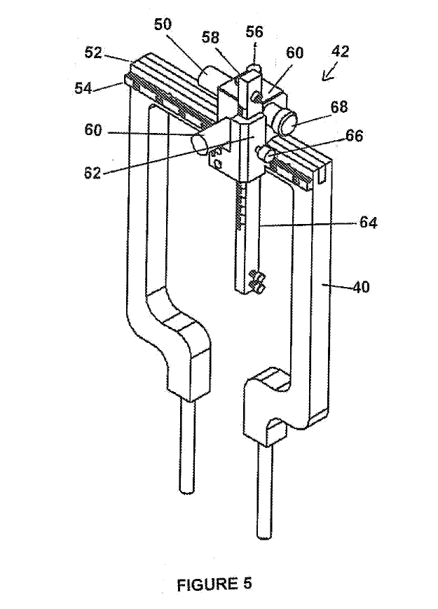

The MIFAT device 32 has two separate linear joints 42 to implement horizontal and vertical movements by manually, respectively. FIG. 5 shows the structure of the tool linear joints with the frame 40 of the MIFAT tool 32. The horizontal joint consists of a multi-turn potentiometer 60 operably connected to an anti-backless spur gear and a rack 52. A linear guide unit 54 is operably connected to the rack and a thumb-screw 56 for locking and a knob are operably connected to the joint. The vertical joint consists of a multi-turn potentiometer (SMT 10/5) 50 is operably connected to an anti-backless spur gear and a rack, 64. A thumb screw for locking 66 and a knob 68 are operably connected to the joint.

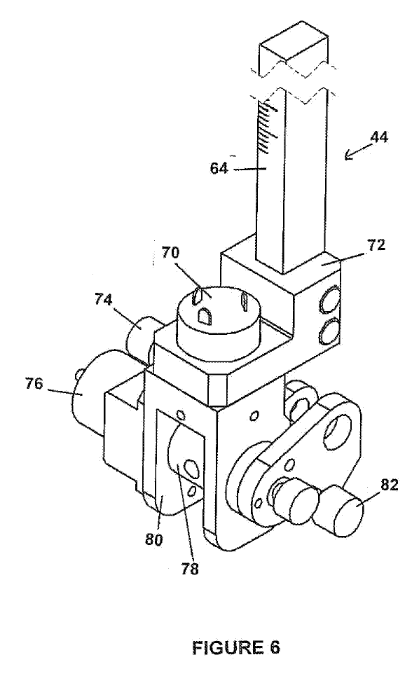

The MIFAT device 32 also has two rotational joints 44: Pan (rotation in horizontal plane) and Tilt (rotation in vertical plane), shown in FIG. 6. The Pan joint unit consists of a rotary potentiometer 70 operably connected to a pan joint support 72, and a locking thumb-screw 74 is operably connected to the joint. The Tilt joint is composed of a rotary potentiometer 76 operably connected to a shaft 78. A tilt joint support 80 and a locking thumb-screw 82 are operably connected.

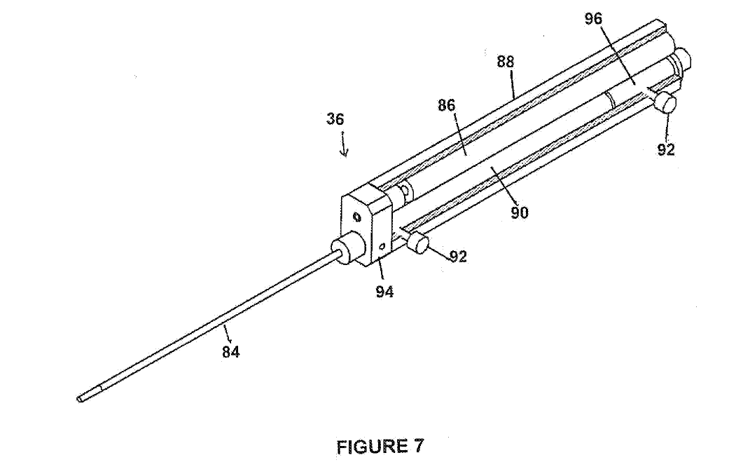

The medical instrument assembly 36 is shown in FIG. 7. The assembly 36 includes a (manual medical tool (needle) 84 is operably connected to a linear potentiometer 86. A body 88 has a guiding hollow shaft 90 for receiving the needle tool 84 which slides therein. Two locking thumb-screws 92 are operably connected to a slide block 94 and a connector 96 separately.

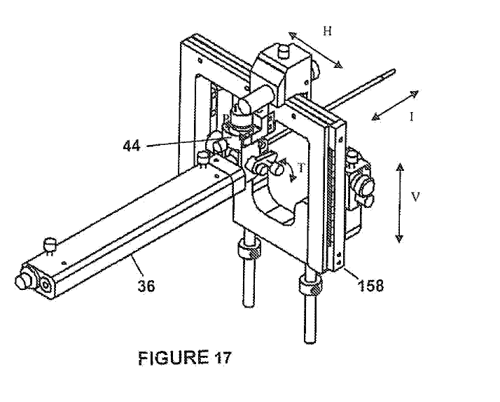

An alternative embodiment of the manual instrumented focal ablation tool constructed in accordance with the present invention is shown in FIG. 17 and FIG. 18 at 158. Only those features which are different from MIFAT device 32 will be discussed. The remaining features are common to both embodiments.

As seen in FIG. 17, the alternative MIFAT 158 is for use in association with an instrument assembly 36. The alternative MIFAT device 158 similarly includes a horizontal translation unit, a vertical translation unit, pan unit, tilt unit and needle penetration unit. The pan unit and tilt unit are the two rotational joints 44 described above. The instrument assembly 36 described above includes a needle penetration unit. FIG. 18 shows the horizontal translation unit and vertical translation unit the alternative MIFAT device 158 which includes frames 160, 161 The horizontal translation unit 162 is essentially the same as horizontal portion of the linear motion joint 42. The vertical translational unit or joint consists of a rack 163, two anti-backlash spur gears and a potentiometer 164, two linear guides units 165 attached on the frames, and a thumb-screw 166 for locking, as well as knob 167 operably connected to the joint.

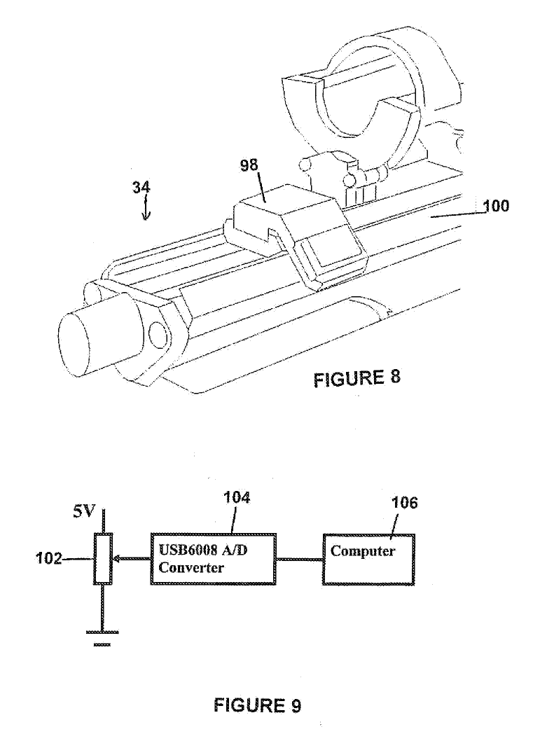

In order to track the ultra-sound probe insertion depth during the procedure, a linear sensor 98 and a linear scale 100 are mounted on stepper 34 as shown on FIG. 8.

Because the MIFAT is mounted mechanically on the stepper 34 (see FIG. 3), and the stepper is electronically encoded, the probe insertion depth with respect to stepper base and the MIFAT frame 40 is always identified on computer. Thus, the needle can be calibrated directly to the TRUS image. The MIFAT and the TRUS probe are secured in a precision stepper interfaced to a computer that stores prostate and tumor images overlaid to the ultrasonic images. They are attached on a precision stabilizer mounted on the operating room (OR) table, as used in standard prostate brachytherapy procedures.

The manual medical tool is spatially registered to the ultrasound images. The real-time ultrasound images are transferred onto a computer that is also situated in the operating room.

The Software of MIFAT implements the following functions:
1. The software displays the live image generated by the trans-rectal ultrasound device being used to image the manual medical tool placement.
2. The software superimposes on the ultrasound image the contours of the treatment target, which will consist of the 3D volumes of the prostate and tumour, which will have been identified on pre-treatment MRI scans.
3. The software calculates and displays the best insertion path for a given target volume.
4. The software specifies the medical instrument assembly settings in order achieve the best insertion path calculated for the target.
   As the manual medical tool is being inserted, the software provides a measure of how close the actual tool insertion path is to the best tool insertion path. The software indicates to the clinician when the tool has arrived at the desired position.

Potentiometers 102 are used to measure each position of needle on x, y, pan, tilt and also the penetration of the needle. The diagram is shown in FIG. 9. Potentiometer 102 is operably connected to an analog to digital (A/D) converter 104. Preferably the A/D converter is a USB6008 A/D converter device from National Instrument. By measuring the output voltage of the potentiometers 102, software 106 will get the positions of the needle and the tip related to the frame of the MIFAT.

To display the real time ultrasound video from the ultrasound machine, MIFAT software captures the video out from the machine using a pinnacle 510-USB video capturer. To implement the video capturing, DirectShow™ technology is used. A class named CDSControl™ is built. There are more than 30 functions in this class to implement the capturing, filtering, overlaying and displaying for the video.

For the contour display, the VTK and DirectShow is used together. The Visualization Toolkit (VTK)™ is an open-source, freely available software system for 3D computer graphics, image processing, and visualization used by thousands of researchers and developers around the world. VTK may be used to produce the contour of the prostate and tumour. Preferably, first vtkSTLReader™ is used to read the 3D model of the tumour and prostate from the STL file (Note: “stl” is derived from the word “Stereolithography.” a stl file is a format used by Stereolithography software to generate information needed to produce 3D models on Stereolithography machines). Secondly a vtkPlane™ is used to define the current image plane based on the measure. Then a vtkcutter™ cut the 3D model to et a set of points which define the contour of prostate and tumour. Finally the two contours are overlaid on the realtime video using DirectShow.

Best path means a line through which a needle should go through and get a best treatment result. This requires the user to input the PTV (Planning Target Volume) as a binary mask, as well as an initial angle to optimize for, and constraints on the angles. The algorithm will determine the distance from a line at a given angle (with the centroid of the PTV being a point on that line), to each of the points in the PTV. The least squares sum of this distance is then minimized. This is implemented in the function get Initial Insertion Angle.

The best path may be determined in light of specific internal (anatomical) structures that the surgeon wishes to avoid. As well or in addition the best path may be determined in light of the volume of the tumor and the most effective path of a laser to the tumor.

Preferably the image area is on top left of the screen. The image in this area is captured from the real time video output of the TRUS unit, and the virtual contours of prostate and cancer are overlaid on the image.

A marker for “aiming” to the target is overlaid on the image. It can help the physician to aim the needle to the target before needle penetration based on the feedback from sensors. The marker indicates the predicted position of the needle tip when it reaches the transverse plane through the target. In order to remind the physician of the relative position of the tip of the needle, one of three statuses is shown on the image:
When the tip is approaching the plane, the color of the marker is green and the shape of the marker is square;
When the tip is within ±2 mm of the plane, the color of the marker is yellow and the shape is a star; and
When tip passes the plane, the color of the marker is red and the shape is a triangle.

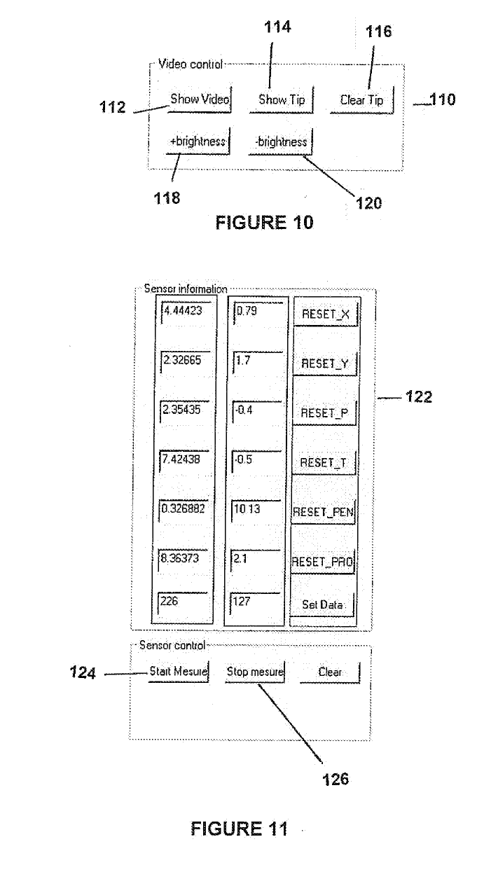

Preferably the video control area 110 is on the right of the screen. A sample video control area 110 is shown in FIG. 10. Preferably, there are five buttons in the area, specifically:
‘Show video’ button: start the video capture 112;
‘Show Tip’ 114 & ‘Clear Tip’ 116 buttons: make the “aiming” marker visible or invisible;
‘+ brightness’ 118 & ‘− brightness’ 120 buttons: increase or decrease by 3% the brightness of the video image

Preferably, the sensor information area 122 can control and show the information from the sensors, as shown in FIG. 11. The ‘Start Measure’ 124 & ‘Stop Measure’ 126 buttons control the sensing procedure. The results are displayed in the text boxes. The text boxes are the voltage signals from sensors; they are reference for instrument engineer. The text boxes show the measurements in millimetre or degree, which are x, y pan, tilt, penetration and motion of the probe, respectively. The physician can view the position and orientation of the needle tip. Other buttons are for calibration purpose; usually the physician does not use them.

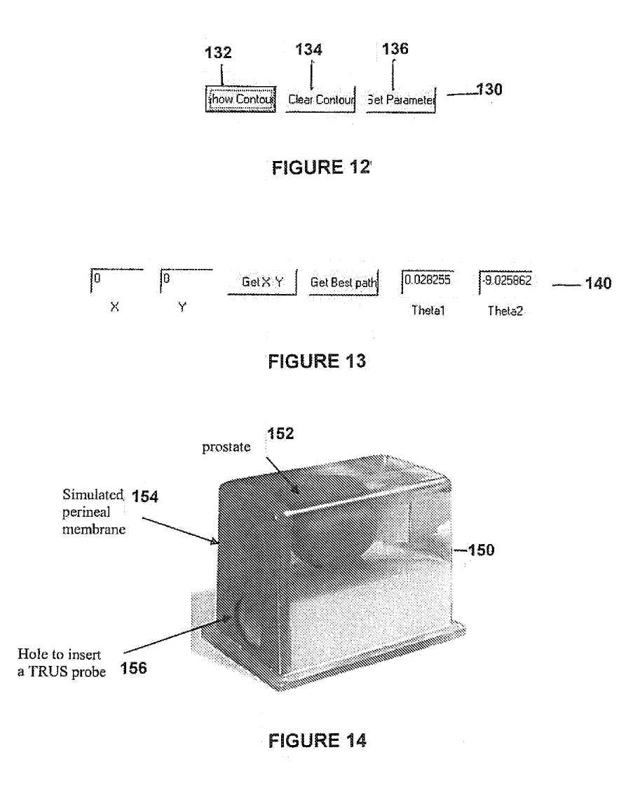

Preferably, as shown in FIG. 12, the contour overlay area 130 reads the 3D model and enable/disable the overlay: “Show Contour” 132 button reads the predefined 3D model of the prostate and target and enable the overlay. “Clear Contour” 134 button can disable the overlay and clear the contour on the screen. “Set parameter” 136 is for debugging purpose.

Preferably, as shown in FIG. 13, the best path area 140 provides the angles for best path from the predefined “mask” file. The “best path” here means a line in the space which stretches from the Entry (A point which needle will start penetrate to target from) through to the Target. The needle path is to follow this line. In MIFAT best path means the position and the orientation—a set of X, Y, Pan and Tilt at the Entry. The “Get best path” button calls a Matlab environment in the background to run Best path software to get the orientation (i.e., P, T) of the best path. Then clicking “Get X Y” button generates the (X, Y) of the entry.

Emulating experiments were designed with a prostate training phantom to demonstrate the MIFAT system. Three major issues for the experiments are described as below.

A commercial prostate training phantom 150 (CIRS Model 053A, shown in FIG. 14 is a view of a portion of the video screen which includes the best path area. The prostate 152 (4 cm×4.5 cm×4 cm) along with structures simulating the rectal wall, seminal vesicles and urethra is contained within an 11.5 cm×7.0 cm×9.5 cm clear acrylic container. Three 0.5 cc lesions are embedded in the prostate. A 3 mm simulated perineal membrane 154 enables various probes and surgical needles to be inserted into the prostate. In one wall of the container there is one 30 mm diameter hole to insert a TRUS probe and one 50 mm diameter hole to insert needles. The possible locations and angles of needle insertion were constrained by the circular hole 156 on the wall of the phantom. The prostate and the lesions of the phantom were traced on pre-operative MR images and provided to the MIFAT software as 3D structures defined using the standardized Stereolithography (STL) format for MRI/TRUS fusion.

For emulating experiments, the prostate phantom 150, the stepper and the tool device were rigidly attached to the base support. Because the tool device was mounted mechanically on the TRUS stepper, and the stepper was electronically encoded, the probe insertion depth with respect to stepper base and the tool frame was always identified on computer. Thus, the needle could be calibrated directly to the TRUS image.

Needle insertion and tracking: The goal was to demonstrate the placement of needle into the phantoms and the integration with the rest of the intra-operative system, especially with real-time ultrasound tracking.

The following sequences were executed in every needle insertion experiment:
1. Set up and calibrate the system;
2. MRI-TRUS image manual fusion, contours overly display on the screen of the computer;
3. Created a best path of the needle insertion;
4. Locate and orient the needle holder and lock the needle up
5. Manually penetrated the needle into the selected target by using the tool device;
6. Locate the needle in real-time ultrasound and computer-display; and
7. Estimate the position error after the needle was inserted.

The purpose of calibration was to determine the parameters which defined the transformation of a point in one coordinate system (i.e. an image) to another coordinate system. For MIFAT system, the real-time (or intra-operative) TRUS image had to be matched to the preoperative MR image so the needle tip could be accurately located according to the best path plan. And the needle tip had to be transformed to the fixed base frame.

The calibration procedure had the following components: manually positioning the TRUS probe so the real-time (or intra-operative) image shown on the computer-based User Interface was similar to the corresponding 2D contours—overlays, which were sliced on the prostate and lesions 3D model that were created with the pre-operative MR (or TRUS, just for the phantom experiments) images; registering the TRUS images to the needle guide via adjusting the mounting position of the phantom and the tool device.

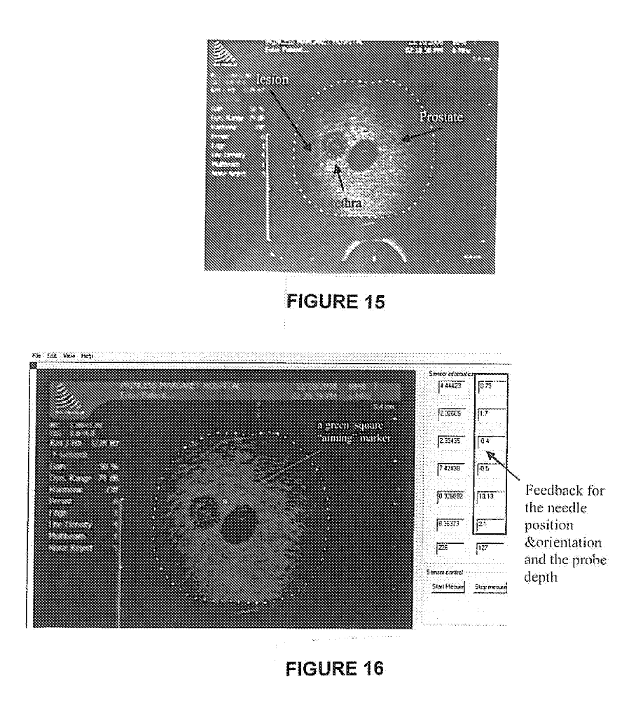

The computer displayed a live 2D—prostate image on top left of its screen. The image was captured from the real-time video output of the TRUS machine, and the MRI-based virtual contours of the prostate were superimposed in green and the contours of the lesions were overlaid on the image. FIG. 15 shows a computer-based image for display of the fused MRI/TRUS data sets. It shows the live 2D-TRUS image (transverse view) with contouring of the prostate and contouring of the lesion. Preferably these are depicted in different colours.

Fused MRI/TRUS guidance needle intervention tracking tests were performed several times.

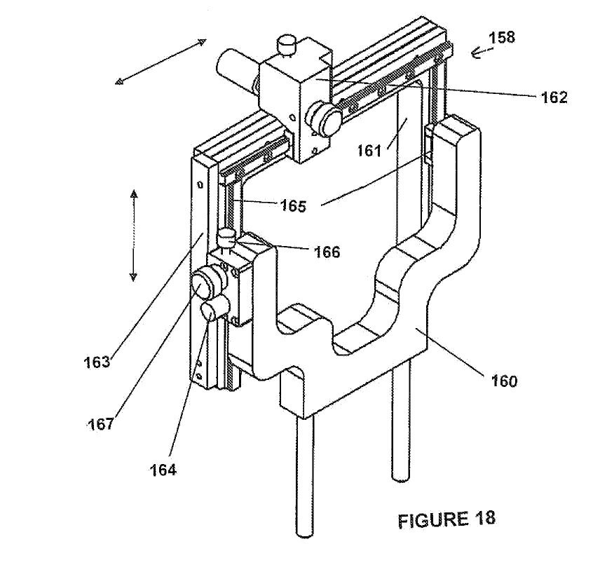

After manually moving the horizontal (X), vertical (Y), Pan and Tilt joints of the tool to the corresponding Entry coordinate created by the best path planning software, (While moving each joint, its displacement was being fed back to computer and shown in the corresponding test box of computer-based User Interface; also a green square “aiming” marker was shown on the image area, as shown in FIG. 16, the needle was manually inserted into the phantom, (visual feedback of the needle tip insertion was being shown on the TRUS image and computer-based User Interface), until the needle tip artifact appears as a high intensity flash near the target, and in the meantime, the color of the “aiming” marker overlaid on the “target” become yellow.

Several experiments on a phantom have shown the capacity of MIFAT to reach its target with a few millimetres accuracy.

The experiments for emulating TRUS-guided interventions on a phantom have demonstrated the feasibility of the MIFAT concept, with fusing preoperative MR images to intra-operative TRUS image and resulting needle intervention accuracies estimated within the acceptable range of a few millimetres. It will likely improve the target accuracy in the future work.

For clinical practices (especially, at the stage of early prostate cancer), the 3D model of prostate and tumor should be created with the pre-operative MR images.

It will be appreciated by those skilled in the art that MIFAT could be used for other minimally invasive surgery such as brachy, biopsy and ablation. As well, the device could be used conjunction with other medical instrument assemblies in other surgical procedures. In addition, it will be appreciated by those skilled in the art that the MIFAT could also be used in association with a magnetic resonance imager (MRI). If MIFAT is used with an MRI the medical instrument assembly position and best path will be shown on the MR image as the medical instrument is being positioned in the patient.

Generally speaking, the systems described herein are directed to the MIFAT device. As required, embodiments of the present invention are disclosed herein. However, the disclosed embodiments are merely exemplary, and it should be understood that the invention may be embodied in many various and alternative forms. The Figures are not to scale and some features may be exaggerated or minimized to show details of particular elements while related elements may have been eliminated to prevent obscuring novel aspects. Therefore, specific structural and functional details disclosed herein are not to be interpreted as limiting but merely as a basis for the claims and as a representative basis for teaching one skilled in the art to variously employ the present invention. For purposes of teaching and not limitation, the illustrated embodiments are directed to a MIFAT device and the MIFAT system.

As used herein, the terms “comprises” and “comprising” are to be construed as being inclusive and opened rather than exclusive. Specifically, when used in this specification including the claims, the terms “comprises” and “comprising” and variations thereof mean that the specified features, steps or components are included. The terms are not to be interpreted to exclude the presence of other features, steps or components.

What is claimed as the invention is:

1. A medical device for use in association with a medical image of a gland/organ having a known reference point, the medical device comprising;
a mechanical frame being positioned at a predetermined location relative to the medical image reference point;
a horizontal joint operably connected to a horizontal position sensor and operably connected to the frame;
a vertical joint operably connected to a vertical position sensor and operably connected to the frame;
a pan joint operably connected to a pan position sensor and operably connected to the frame;
a tilt joint operably connected a tilt position sensor and operably connected to the frame;
a medical instrument assembly operably connected to a medical instrument position sensor and operably connected to the horizontal joint, the vertical joint, the pan joint and the tilt joint; and
a control system operably connected to the horizontal position sensor, the vertical position sensor, the pan position sensor, the tilt position sensor, the tilt position sensor and the medical instrument position sensor whereby the control system determines an actual position of a predetermined location on the medical instrument assembly relative to the frame.

2. The medical device as claimed in claim 1 further including a mover being positioned at a predetermined location relative to the medical image reference point, wherein the frame is movably attached to the mover and further including a means for determining the position of the frame relative to the mover such that the position of the frame is positioned at a predetermined location relative to the medical image reference point.

3. The medical device as claimed in claim 2 wherein the horizontal joint and horizontal position sensor include a multi-turn potentiometer operably connected to an anti-backless spur gear and a rack, a linear guide unit operably connected to the rack, a locking mechanism operably connected to the rack and a means for moving the rack operably connected to the rack.

4. The medical device as claimed in claim 3 wherein the vertical joint and vertical position sensor include a multi-turn potentiometer operably connected to an anti-backless spur gear and a rack, a locking mechanism operably connected to the rack and a means for moving the rack operably connected to the rack.

5. The medical device as claimed in claim 4 wherein the pan joint and pan position sensor includes a rotary potentiometer a pan joint support operably connected to the potentiometer and a locking mechanism operably connected to the potentiometer.

6. The medical device as claimed in claim 5 wherein the tilt joint and tilt position sensor includes a rotary potentiometer a shaft operably connected to the potentiometer, a tilt joint support operably connected to the potentiometer and a locking mechanism.

7. The medical device as claimed in claim 6 wherein the medical instrument assembly is a needle assembly.

8. The medical device as claimed in claim 7 wherein the needle assembly and medical instrument assembly position sensor includes a linear potentiometer, a needle tool operably connected to the linear potentiometer, a guiding shaft for receiving the needle tool, a lock operably connected to the guiding shaft, a slide block operably connected to the guiding shaft and a connector.

9. The medical device as claimed in claim 8 wherein the medical image is one of an ultrasound image and a MR image.

10. The medical device as claimed in claim 9 wherein the medical image is obtained in real time.

11. The medical device as claimed in claim 8 wherein the medical image is a blended real time ultrasound image and a pre-operative MR image.

12. The medical device as claimed in claim 1 further including a mover being positioned at a predetermined location relative to the medical image reference point, wherein the frame is movably attached to the mover and further including a means for determining the position of the frame relative to the mover such that the position of the frame is positioned at a predetermined location relative to the medical image reference point.

13. The medical device as claimed in claim 1 wherein the horizontal joint and horizontal position sensor include a multi-turn potentiometer operably connected to an anti-backless spur gear and a rack, a linear guide unit operably connected to the rack, a locking mechanism operably connected to the rack and a means for moving the rack operably connected to the rack.

14. The medical device as claimed in claim 1 wherein the vertical joint and vertical position sensor include a multi-turn potentiometer operably connected to an anti-backless spur gear and a rack, a locking mechanism operably connected to the rack and a means for moving the rack operably connected to the rack.

15. The medical device as claimed in claim 1 wherein the pan joint and pan position sensor includes a rotary potentiometer a pan joint support operably connected to the potentiometer and a locking mechanism operably connected to the potentiometer.

16. The medical device as claimed in claim 1 wherein the tilt joint and tilt position sensor includes a rotary potentiometer a shaft operably connected to the potentiometer, a tilt joint support operably connected to the potentiometer and a locking mechanism.

17. The medical device as claimed in claim 1 wherein the medical instrument assembly is a needle assembly.

18. The medical device as claimed in claim 17 wherein the needle assembly and medical instrument assembly position sensor includes a linear potentiometer, a needle tool operably connected to the linear potentiometer, a guiding shaft for receiving the needle tool, a lock operably connected to the guiding shaft, a slide block operably connected to the guiding shaft and a connector.

19. The medical device as claimed in claim 1 wherein the medical image is one of an ultrasound image and a MR image.

20. The medical device as claimed in claim 1 wherein the medical image is obtained in real time.

21. The medical device as claimed in claim 1 wherein the medical image is a blended real time ultrasound image and a pre-operative MR image.

22. A method of positioning a medical instrument assembly including a medical instrument comprising the steps of:
obtaining a magnetic resonance image;
obtaining an ultrasound image;
merging the magnetic resonance image with the ultrasound image to obtain a merged image;
determining a position of a predetermined point on the medical instrument assembly; and
locating the position of the predetermined point on the merged image.

23. The method as claimed in claim 22 wherein the position of the predetermined point of the medical instrument is being determined continuously in real time and a location of the point moves on the merged image as the medical instrument assembly moves.

24. The method as claimed in claim 23 wherein ultrasound image is being obtained continuously in real time.

25. The method as claimed in claim 22 further including the step of determining a best path to reach a predetermined target in order to move the medical instrument and show the best path on the merged image.

26. A method of positioning a medical instrument assembly including a medical instrument comprising the steps of:
obtaining a magnetic resonance image;
determining a position of a predetermined point on the medical instrument assembly connected to a manual medical tool system; and
locating the position of the predetermined point on the magnetic resonance image.

27. The method as claimed in claim 26 wherein the position of the predetermined point of the medical instrument is being determined continuously in real time and a location of the point moves on the magnetic resonance image as the medical instrument assembly moves.

28. The method as claimed in claim 27 wherein magnetic resonance image is being updated as the medical instrument is being moved.

29. The method as claimed in claim 26 further including the step of determining a best path based to move the medical instrument and showing the best path on the magnetic resonance image.

30. The method as claimed in claim 22 wherein the method is used in association with minimally invasive surgery.

31. The method as claimed in claim 30 wherein the minimally invasive surgery is chosen from the group consisting of focal ablation, brachytherapy and biopsy.

32. The method as claimed in claim 26 wherein the method is used in association with minimally invasive surgery.

33. The method as claimed in claim 30 wherein the minimally invasive surgery is chosen from the group consisting of focal ablation, brachytherapy and biopsy.
